(19) United States
(12) Patent Application Publication
(10) Pub. No.: US 2025/0369970 A1
(43) Pub. Date: Dec. 4, 2025

(54) RECOMBINANT ANTIBODIES, KITS COMPRISING THE SAME, AND USES THEREOF IN DIAGNOSING AFRICAN SWINE FEVER VIRUS

(71) Applicant: Academia Sinica, Taipei (TW)

(72) Inventors: An-Suei YANG, Taipei (TW); Chung-Ming YU, Taipei (TW); Chao-Ping TUNG, Taipei (TW); Hung-Ju HSU, Taipei (TW); Hung-Pin PENG, Taipei (TW); Chi-Yung CHEN, Taipei (TW); Yu-Chuan HUANG, Taipei (TW); Pei-Hsun TSAI, Taipei (TW); Szu-Yu LIN, Taipei (TW); Fei-Hung HUNG, Taipei (TW)

(21) Appl. No.: 18/867,443

(22) PCT Filed: May 20, 2022
(86) PCT No.: PCT/US2022/030318

§ 371 Date: Nov. 20, 2024

(51) Int. Cl. G01N 33/569; C07K 16/08

(52) U.S. Cl. CPC G01N 33/56983; C07K 16/081; C07K 2317/56; C07K 2317/92; G01N 2333/01

(57) ABSTRACT

Disclosed herein are recombinant antibodies or the fragments thereof for detecting African swine fever virus (ASFV). Also disclosed herein are kits comprising the recombinant antibodies, and methods of diagnosing the infection of ASFV by using the recombinant antibody or kit.

### BACKGROUND OF THE INVENTION

### 1. Field of the Invention

The present disclosure in general relates to the field of disease diagnosis. More particularly, the present disclosure relates to recombinant antibodies specific to African swine fever virus (ASFV), and uses thereof in diagnosing ASFV infection.

### 2. Description of Related Art

ASFV is a large, enveloped, double-stranded DNA virus belonging to the genus Asfivirus, and the only member of the family Asfarviridae. It is the causative agent of African swine fever (ASF), a highly contagious and severe hemorrhagic viral disease of domestic pigs. The first recorded ASF outbreaks were reported in Kenya in 1914. The disease continues to spread throughout Europe since 2007, and was reported in East Asia (including China, Philippines, Vietnam, South Korea and East Timor) in 2019. ASF is associated with high economic losses, and is listed by the World Organization for Animal Health as the highest priority disease of domestic pigs in many affected countries. There is currently no vaccine for the control of ASF. The approaches available for control rely on early detection of cases followed by stamping out and enforcement of strict biosecurity measures to prevent further spread.

In view of the forging, there exists in the related art a need for an agent and/or a method for detecting the ASFV infection at an early stage so as to prevent and control the spread of the disease in time.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a recombinant antibody or the fragment thereof. According to embodiments of the present disclosure, the recombinant antibody or the antibody fragment comprises a light chain variable (VL) domain and a heavy chain variable (VH) domain, in which the VL domain comprises a first light chain complementarity determining region (CDR-L1), a second light chain CDR (CDR-L2), and a third light chain CDR (CDR-L3); and the VH domain comprises a first heavy chain CDR (CDR-H1), a second heavy chain CDR (CDR-H2), and a third heavy chain CDR (CDR-H3).

According to some embodiments of the present disclosure, the CDR-L1, CDR-L2 and CDR-L3 respectively have the amino acid sequences of SEQ ID NOs: 1-3, and the CDR-H1, CDR-H2 and CDR-H3 respectively have the amino acid sequences of SEQ ID NOs: 4-6. According to some preferred embodiments, the VL domain comprises an amino acid sequence at least 85% identical to SEQ ID NO: 37, and the VH domain comprises an amino acid sequence at least 85% identical to SEQ ID NO: 38. In some working examples, the VL and VH domains of the recombinant antibody or antibody fragment respectively comprise amino acid sequences 100% identical to SEQ ID NOs: 37 and 38.

According to certain embodiments of the present disclosure, the CDR-L1, CDR-L2 and CDR-L3 respectively have the amino acid sequences of SEQ ID NOs: 7-9, and the CDR-H1, CDR-H2 and CDR-H3 respectively have the amino acid sequences of SEQ ID NOs: 10-12.According to some preferred embodiments, the VL domain comprises an amino acid sequence at least 85% identical to SEQ ID NO: 39, and the VH domain comprises an amino acid sequence at least 85% identical to SEQ ID NO: 40. In some working examples, the VL and VH domains of the recombinant antibody or antibody fragment respectively comprise amino acid sequences 100% identical to SEQ ID NOs: 39 and 40.

According to certain embodiments of the present disclosure, the CDR-L1, CDR-L2 and CDR-L3 respectively have the amino acid sequences of SEQ ID NOs: 13-15, and the CDR-H1, CDR-H2 and CDR-H3 respectively have the amino acid sequences of SEQ ID NOs: 16-18. According to some preferred embodiments, the VL domain comprises an amino acid sequence at least 85% identical to SEQ ID NO: 41, and the VH domain comprises an amino acid sequence at least 85% identical to SEQ ID NO: 42. In certain examples, the VL and VH domains of the recombinant antibody or antibody fragment respectively comprise amino acid sequences 100% identical to SEQ ID NOs: 41 and 42.

According to some embodiments of the present disclosure, the CDR-L1, CDR-L2 and CDR-L3 respectively have the amino acid sequences of SEQ ID NOs: 19-21, and the CDR-H1, CDR-H2 and CDR-H3 respectively have the amino acid sequences of SEQ ID NOs: 22-24. According to the preferred embodiments, the VL domain comprises an amino acid sequence at least 85% identical to SEQ ID NO: 43, and the VH domain comprises an amino acid sequence at least 85% identical to SEQ ID NO: 44. In certain examples, the VL and VH domains of the recombinant antibody or antibody fragment respectively comprise amino acid sequences 100% identical to SEQ ID NOs: 43 and 44.

According to some embodiments of the present disclosure, the CDR-L1, CDR-L2 and CDR-L3 respectively have the amino acid sequences of SEQ ID NOs: 25-27, and the CDR-H1, CDR-H2 and CDR-H3 respectively have the amino acid sequences of SEQ ID NOs: 28-30. According to some preferred embodiments, the VL domain comprises an amino acid sequence at least 85% identical to SEQ ID NO: 45, and the VH domain comprises an amino acid sequence at least 85% identical to SEQ ID NO: 46. In certain working examples, the VL and VH domains of the recombinant antibody or antibody fragment respectively comprise amino acid sequences 100% identical to SEQ ID NOs: 45 and 46.

According to some embodiments of the present disclosure, the CDR-L1, CDR-L2 and CDR-L3 respectively have the amino acid sequences of SEQ ID NOs: 31-33, and the CDR-H1, CDR-H2 and CDR-H3 respectively have the amino acid sequences of SEQ ID NOs: 34-36. According to some preferred embodiments, the VL domain comprises an amino acid sequence at least 85% identical to SEQ ID NO: 47, and the VH domain comprises an amino acid sequence at least 85% identical to SEQ ID NO: 48. In some specific examples, the VL and VH domains of the recombinant antibody or antibody fragment respectively comprise amino acid sequences 100% identical to SEQ ID NOs: 47 and 48.

Another aspect of the present disclosure is directed to a kit for detecting the presence of ASFV in a biological sample. The kit comprises a first recombinant antibody, a second recombinant antibody, and a container containing the first and second recombinant antibodies, in which the first and second recombinant antibody are independently selected from the recombinant antibodies as described in the first aspect of the present disclosure. According to certain embodiments, one of the first and second recombinant antibodies serves as a capture antibody, and the other of the first and second recombinant antibodies serves as a detection antibody for use in a detection technique, e.g., an enzyme-linked immunosorbent assay (ELISA), lateral flow immunoassay (LFIA), and western blotting (WB) assay.

According to some embodiments, the CDR-LI, CDR-L2, CDR-L3, CDR-H1, CDR-H2 and CDR-H3 of the first recombinant antibody respectively comprise the amino acid sequences of SEQ ID NOs: 1-6, and the CDR-L1, CDR-L2, CDR-L3, CDR-H1, CDR-H2 and CDR-H3 of the second recombinant antibody respectively comprise the amino acid sequences of SEQ ID NOs: 31-36. In certain exemplary embodiments, the VL and VH domains of the first recombinant antibody respectively comprise amino acid sequences at least 85% identical to SEQ ID NOs: 37 and 38, and the VL and VH domains of the second recombinant antibody respectively comprise amino acid sequences at least 85% identical to SEQ ID NOs: 47 and 48. In one specific example, the VL and VH domains of the first recombinant antibody respectively comprise amino acid sequences 100% identical to SEQ ID NOs: 37 and 38, and the VL and VH domains of the second recombinant antibody respectively comprise amino acid sequences 100% identical to SEQ ID NOs: 47 and 48.

According to certain embodiments, the CDR-L1, CDR-L2, CDR-L3, CDR-H1, CDR-H2 and CDR-H3 of the first recombinant antibody respectively comprise the amino acid sequences of SEQ ID NOs: 7-12, and the CDR-L1, CDR-L2, CDR-L3, CDR-H1, CDR-H2 and CDR-H3 of the second recombinant antibody respectively comprise the amino acid sequences of SEQ ID NOs: 25-30. In certain exemplary embodiments, the VL and VH domains of the first recombinant antibody respectively comprise amino acid sequences at least 85% identical to SEQ ID NOs: 39 and 40, and the VL and VH domains of the second recombinant antibody respectively comprise amino acid sequences at least 85% identical to SEQ ID NOs: 45 and 46. In one specific example, the VL and VH domains of the first recombinant antibody respectively comprise amino acid sequences 100% identical to SEQ ID NOs: 39 and 40, and the VL and VH domains of the second recombinant antibody respectively comprise amino acid sequences 100% identical to SEQ ID NOs: 45 and 46.

According to alternative embodiments, the CDR-L1, CDR-L2, CDR-L3, CDR-H1, CDR-H2 and CDR-H3 of the first recombinant antibody respectively comprise the amino acid sequences of SEQ ID NOs: 13-18, and the CDR-L1, CDR-L2, CDR-L3, CDR-H1, CDR-H2 and CDR-H3 of the second recombinant antibody respectively comprise the amino acid sequences of SEQ ID NOs: 19-24. In certain exemplary embodiments, the VL and VH domains of the first recombinant antibody respectively comprise amino acid sequences at least 85% identical to SEQ ID NOs: 41 and 42, and the VL and VH domains of the second recombinant antibody respectively comprise amino acid sequences at least 85% identical to SEQ ID NOs: 43 and 44. In one specific example, the VL and VH domains of the first recombinant antibody respectively comprise amino acid sequences 100% identical to SEQ ID NOs: 41 and 42, and the VL and VH domains of the second recombinant antibody respectively comprise amino acid sequences 100% identical to SEQ ID NOs: 43 and 44.

Also provided herein is a method of determining whether a subject is infected by ASFV via a biological sample isolated from the subject. The method comprises the steps of, detecting the presence or absence of a viral protein of ASFV in the biological sample by use of the antibody fragment, the recombinant antibody or the kit of the present disclosure, wherein the presence of the viral protein indicates that the subject is infected by the ASFV.

According to some embodiments, the viral protein is 30-kDa phosphoprotein (p30 protein).

Based on the result, a skilled artisan or a clinical practitioner may administer to a subject in need thereof (e.g., an ASFV-infected swine) an appropriate treatment in time. Specifically, in the case when the viral protein (e.g., p30 protein) is present in the biological sample of a subject, then an effective amount of an anti-viral treatment (e.g., apigenin, enrofloxacin, grepafloxacin, balofloxacin, tosufloxacin, gatifloxacin, garenoxacin, or a combination thereof) is administered to the subject so as to alleviate and/or ameliorate the symptoms associated with the ASFV infection. Alternatively, the ASFV-infected swine is isolated from other livestock on the farm so as to prevent and control the spread of ASFV.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIGS. 1A to 1C are photographs respectively depicting the binding affinity and specificity of specified antibody pairs to p30 protein of ASFV according to Example 2 of the present disclosure, in which the p30 protein was expressed by HEK293 cells. FIG. 1A: The binding affinity (Panel (A)) and binding specificity (Panel (B)) of D96C77 antibody pair to p30 protein. FIG. 1B: The binding affinity (Panel (A)) and binding specificity (Panel (B)) of D91C90 antibody pair to p30 protein. FIG. 1C: The binding affinity (Panel (A)) and binding specificity (Panel (B)) of D97C52 antibody pair to p30 protein. The detection limit was marked by symbol “*” in Panel (A) of FIGS. 1B and 1C. The D96C77 antibody pair comprises p30-077 IgG as the capture antibody and p30-096 IgG as the detection antibody; the D91C90 antibody pair comprises p30-090 IgG as the capture antibody and p30-091 IgG as the detection antibody; and the D97C52 antibody pair comprises p30-052 IgG as the capture antibody and p30-097 IgG as the detection antibody.

FIGS. 2A to 2C are photographs respectively depicting the binding affinity and specificity of specified antibody pairs to p30 protein of ASFV according to Example 2 of the present disclosure, in which the p30 protein was expressed by Escherichia coli (E. coli). FIG. 2A: The binding affinity (Panel (A)) and binding specificity (Panel (B)) of D96C77 antibody pair to p30 protein. FIG. 2B: The binding affinity (Panel (A)) and binding specificity (Panel (B)) of D91C90 antibody pair to p30 protein. FIG. 2C: The binding affinity (Panel (A)) and binding specificity (Panel (B)) of D97C52 antibody pair to p30 protein. The detection limit was marked by symbol “*” in Panel (A) of FIGS. 2B and 2C.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “antibody” is used in the broadest sense and specifically covers monoclonal antibodies (including full length monoclonal antibodies), polyclonal antibodies, multi-specific or multivalent antibodies (e.g., bi-specific antibodies), and antibody fragments so long as they exhibit the desired biological activity. The term “antibody fragment” or “the fragment of an antibody” refers to a portion of a full-length antibody, generally the antigen binding or variable domain (i.e., VL and VH domains) of a full-length antibody. Examples of the antibody fragment include fragment antigen-binding (Fab), Fab′, F(ab′)2, single-chain variable fragment (scFv), diabody, linear antibody, single-chain antibody molecule, and multi-specific antibody formed from antibody fragments.

The term “complementarity determining region (CDR)” used herein refers to the hypervariable region of an antibody molecule that forms a surface complementary to the 3-dimensional surface of a bound antigen. Proceeding from N-terminus to C-terminus, each of the antibody heavy and light chains comprises three CDRs (i.e., CDR-1, CDR-2, and CDR-3). A HLA-DR antigen-binding site, therefore, includes a total of six CDRs that comprise three CDRs from the variable domain of a heavy chain (i.e., CDR-H1, CDR-H2, and CDR-H3), and three CDRs from the variable domain of a light chain (i.e., CDR-L1, CDR-L2, and CDR-L3). The amino acid residues of CDRs are in close contact with bound antigen, wherein the closest antigen contact is usually associated with the heavy chain CDR3.

As used herein, the term “variable domain” or “variable region” of an antibody refers to the amino-terminal regions of heavy or light chain of the antibody. These regions are generally the most variable parts of an antibody and contain the antigen-binding sites. The term “variable” refers to the fact that certain portions of the variable regions differ extensively in sequence among antibodies, and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprises four FR regions, largely adopting a beta-sheet configuration, connected by three CDRs, which form loops connecting, and in some cases forming part of, the beta-sheet structure. The CDRs in each chain are held together in close proximity by the FR regions, and with the CDRs from the other chain, contribute to the formation of the antigen-binding site of antibodies. The constant regions are not involved directly in binding an antibody to an antigen, but exhibit various effector functions, such as participation of the antibody in antibody-dependent cellular toxicity.

“Percentage (%) sequence identity” with respect to any amino acid sequence identified herein is defined as the percentage of amino acid residues in a candidate sequence that are identical with the amino acid residues in the specific reference sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two amino acid sequences was carried out by computer program Blastp (protein-protein BLAST) provided online by Nation Center for Biotechnology Information (NCBI). The percentage sequence identity of a given sequence A to a subject sequence B (which can alternatively be phrased as a given sequence A that has a certain % sequence identity to a given sequence B) is calculated by the formula as follows:

$$\frac{X}{Y} \times 100 \%$$

where X is the number of amino acid residues scored as identical matches by the sequence alignment program BLAST in that program's alignment of A and B, and where Y is the total number of amino acid residues in the subject sequence B.

As discussed herein, minor variations in the amino acid sequences of antibodies are contemplated as being encompassed by the presently disclosed and claimed inventive concept(s), providing that the variations in the amino acid sequence maintain at least 85% sequence identity, such as at least 85%, 86%, 87%, 88%, 89%, 90%, 91%, 92%, 93%, 94%, 95%, 96%, 97%, 98% and 99% sequence identity. Antibodies of the present disclosure may be modified specifically to alter a feature of the peptide unrelated to its physiological activity. For example, certain amino acids can be changed and/or deleted without affecting the physiological activity of the antibody in this study (i.e., the ability of binding to coronavirus). In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the peptide derivative. Fragments or analogs of antibodies can be readily prepared by those of ordinary skill in the art. Preferred amino-and carboxyl-termini of fragments or analogs occur near boundaries of functional regions.

The term “subject” is intended to refer to both the male and female gender unless one gender is specifically indicated.

### II. Description of the Invention

### (II-1) Methods for Selecting an Antibody Fragment Specific to ASFV

The first aspect of the present disclosure is directed to a method for selecting an antibody fragment specific to ASFV. According to embodiments of the present disclosure, the method comprises,
(a) providing a phage-displayed single-chain variable fragment (scFv) library that comprises a plurality of phage-displayed scFvs, wherein the VH domain of each phage-displayed scFvs has a binding affinity to protein A, and the VL domain of each phage-displayed scFvs has a binding affinity to protein L;
(b) exposing the phage-displayed scFv library of the step (a) to a viral protein derived from the ASFV;
(c) selecting, from the phage-displayed scFv library of the step (b), a plurality of phages that respectively express scFvs exhibiting binding affinity to the viral protein;
(d) respectively enabling the plurality of phages selected in the step (c) to express a plurality of soluble scFvs;
(e) exposing the plurality of soluble scFvs of the step (d) to the viral protein;
(f) determining the respective binding affinity of the plurality of soluble scFvs to the viral protein in the step (e); and
(g) based on the results determined in the step (f), selecting one soluble scFv that exhibits superior binding affinity to the viral protein over the other soluble scFvs of the plurality of soluble scFvs as the antibody fragment.

In the step (a), a phage-displayed scFv library is provided. According to the embodiments of the present disclosure, the framework of the phage-displayed scFv library is based on the human IGKV1-NL1*01/IGHV3-23*04 germline sequence, and the complementarity determining region (CDR, including CDR-L1, CDR-L2, CDR-L3, CDR-H1, CDR-H2, and CDR-H3) thereof are diversified by PCR reaction using desired primers. After the selection of protein A and protein L, the phage-displayed scFv library (hereinafter as “GH2 library”) is produced, in which each of the plurality of phage-displayed scFvs has a VH domain capable of binding to protein A, and a VL domain capable of binding to protein L. This phage-displayed scFv library can be constructed using the method described in the co-pending PCT applications, PCT/US2016/19128 and PCT/US2018/56627, and the publication of Ing-Chien Chen et al. (High throughput discovery of influenza virus neutralizing antibodies from phage-displayed synthetic antibody libraries, Scientific Reports 7, Article number: 14455 (2017)). The entirety of the application and publication are incorporated herein by reference.

In the step (b), the GH2 library is exposed to a viral protein derived from the ASFV. According to some embodiments, the viral protein is p30 protein, an early protein as a part of the inner membrane of ASFV particles. In some working examples, the p30 protein of ASFV comprises the amino acid sequence of SEQ ID NO: 49. According to some working examples, the p30 protein is immobilized on a matrix (such as an agarose resin or polyacrylamide) and then mixed with the present GH2 library.

In the step (c), a plurality of phages respectively expressing scFvs that exhibit binding affinity to the viral protein (i.e., p30 protein) are selected from the GH2 library. Specifically, the product of the step (b) is subjected to an elution buffer, which generally is an acidic solution (such as glycine solution, pH 2.2), so as to disrupt the binding between the viral protein and phage-display scFv. By this way, the plurality of phages that respectively express scFvs exhibiting binding affinity to the viral protein are collected.

Optionally, the step (c) is carried out under an acidic condition. Specifically, the product of the step (b) may be subjected to an acidic treatment (for example, a washing buffer having a pH value ranging between 5-7, such as pH 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 or 7; preferably, a washing buffer having a pH value of 5.0) followed by the afore-mentioned elution step to collect the plurality of phages.

Next, in the step (d), the plurality of phages selected in the step (c) are subjected to conditions that enable them to produce a plurality of soluble scFvs. This step can be carried out by using methods known to any person having ordinary skill in the art. According to certain embodiments of the present disclosure, the expression of VH and VL domains may be driven by a lactose operon (lac operon); as known by one skilled artisan, the lac operon would be induced by isopropyl-thio-β-D-galactoside (IPTG), which then drives the expression of the down-stream genes (i.e., genes encoding the VH and VL domains). The produced scFv are then secreted into the supernatant of culture medium and could be collected therefrom.

In the step (e), the soluble scFvs produced in the step (d) are respectively mixed with the viral protein (i.e., p30 protein) so as to form the protein-scFv complexes.

Then, in the step (f), the level of the protein-scFv complexes formed in the step (e) is determined by a method known to a person having ordinary skill in the art for analyzing the binding affinity of two molecules (e.g., the binding affinity of an antibody to an antigen); for example, ELISA, western blotting assay, flow cytometry, surface plasmon resonance (SPR), or LFIA. In general, the level of the protein-scFv complexes is proportional to the binding affinity of the scFv to the viral protein (i.e., p30 protein). According to one working example, the level of the protein-scFv complex (i.e., the binding affinity of the soluble scFv to the vial protein) is determined by ELISA.

Finally, in the step (g), the antibody fragment is selected based on the binding affinity determined in the step (f). More specifically, the soluble scFv that exhibits superior affinity to the viral protein (i.e., p30 protein) over the other soluble scFvs of the plurality of soluble scFvs is selected as the antibody fragment.

According to some embodiments of the present disclosure, 6 antibody fragments, respectively designated as “p30-052 scFv”, “p30-077 scFv”, “p30-090 scFv”, p30-091 scFv ”, “p30-096 scFv”, and “p30-097 scFv”, are selected from different rounds of selection.

### (II-2) Methods for Producing Recombinant Antibodies

The antibody fragment selected from section (II-1) of the present disclosure is useful in the preparation of a recombinant antibody, which structurally comprises a VL domain, a light chain constant (CL) domain, a VH domain and a heavy chain constant (CH) domain. The method of using the selected antibody fragment to produce a recombinant antibody comprises the steps of,
(1) providing a phage that expresses the selected antibody fragment;
(2) extracting a phagemid DNA corresponding to the phage of the step (1);
(3) respectively amplifying a first nucleic acid sequence that encodes a VH domain, and a second nucleic acid sequence that encodes a VL domain by PCR using the phagemid DNA of the step (2) as a template;
(4) inserting the first and second nucleic acid sequences into an expression vector that comprises a third and a fourth nucleic acid sequences, wherein the third nucleic acid sequence encodes the CH domain of an immunoglobulin, and the fourth nucleic acid sequence encodes the CL domain of the immunoglobulin; and
(5) transfecting a host cell with the expression vector of the step (4) that comprises the first, second, third, and fourth nucleic acid sequences so as to produce the recombinant antibody.

In the present method, the phage that expresses the selected antibody fragment is used as a starting material for the preparation of a recombinant antibody (i.e., the step (1)).

Then, the phagemid DNA corresponding to the antibody fragment-expressing phage is extracted as described in the step (2). Depending on intended purposes, the phagemid may be extracted by lysing the phage; alternatively, the phagemid may be obtained from a bacterial clone (i.e., the phagemid-containing bacterial clone). The extraction of phage DNA from the phage or bacterial clone could be achieved via any conventional DNA extraction technique; for example, the phenol/chloroform assay, and detergent (e.g., sodium dodecyl sulfate, TWEEN®-20,NP-40, and TRITON® X-100)/acetic acid assay.

In the step (3), the thus extracted phagemid DNA then serves as a template to respectively amplify the first nucleic acid sequence that encodes the CDR-H1, CDR-H2, and CDR-H3 by PCR using specific primers (forward primer: SEQ ID NO: 50; reverse primer: SEQ ID NO: 51), and the second nucleic acid sequence that encodes the CDR-L1, CDR-L2, and CDR-L3 by PCR using specific primers (forward primer: SEQ ID NO: 52; reverse primer: SEQ ID NO: 53).

In the step (4), the amplified first and second nucleic acid sequences are inserted into an expression vector, which comprises a third nucleic acid sequence encoding the constant regions of the heavy chain of an immunoglobulin, and a fourth nucleic acid sequence encoding the constant regions of the light chain of the immunoglobulin. As could be appreciated, the immunoglobulin can be any of IgG, IgA, IgD, IgE, and IgM. In one preferred embodiment of the present disclosure, the immunoglobulin is IgG. Specifically, the first and second nucleic acid sequences are first linked by a linker, which is amplified from pIgG vector by PCR. According to the embodiment of the present disclosure, the linker comprises in sequence: the CL domain, a bovine growth hormone (BGH) polyadenylation (polyA) signal, a human CMV promoter, and a signal peptide of IgG heavy chain. For the presences of the complementary sequences between the 3′-end of second nucleic acid sequence and the 5′-end of linker, and the complementary sequences between the 3′-end of the linker and the 5′-end of the first nucleic acid sequence, the second nucleic acid sequence, the linker and the first nucleic acid sequence can be assembled in sequence via overlap extension polymerase chain reaction (OE-PCR). The assembled product is then inserted into the expression vector pIgG by use of the restriction enzymes. Structurally, the constructed expression vector comprises in sequence: a first human CMV promoter, a signal peptide of IgG light chain, the second nucleic acid sequence, the CL domain, a first BGH-polyA signal, a second human CMV promoter, a signal peptide of IgG heavy chain, the first nucleic acid sequence, the CH domain, and a second BGH-polyA signal, in which the second nucleic acid sequence and the CL domain are driven by the first human CMV promoter so as to express the light chain of the recombinant antibody, and the first nucleic acid sequence and the CH domain are driven by the second human CMV promoter to express the heavy chain of the recombinant antibody.

In the step (5), the expression vector constructed in step (4) is transfected into a host cell so as to produce the present recombinant antibody. The commonly used host cell is a mammalian cell, such as a HEK293 cell. The transfection can be performed by any method familiar by one skilled artisan, including chemical-based method (e.g., calcium phosphate, liposome, and cationic polymer), non-chemical method (e.g., electroporation, cell squeezing, sonoporation, optical transfection, protoplast fusion, and hydrodynamic delivery), particle-based method (e.g. gene gun, magnetofection, and impalefection), and viral method (e.g., adenoviral vector, sindbis viral vector, and lentiviral vector). The thus-produced recombinant antibody is secreted into the supernatant of the culture medium, and can be purified therefrom by any purification method familiar by any skilled person; for example, the purification can be achieved by affinity binding with protein A or protein G.

According to some embodiments of the present disclosure, 6 recombinant antibodies are produced from the antibody fragments of section (II-1) of the present disclosure, and are designated as “p30-052 IgG”, “p30-077 IgG”, “p30-090 IgG”, “p30-091 IgG”, “p30-096 IgG” and “p30-097 IgG”, respectively.

### (II-3) Recombinant Antibodies or the Fragment Thereof

According to certain embodiments, each of the antibody fragments selected from section (II-1) of the present disclosure (including p30-052, p30-077, p30-090, p30-091, p30-096 and p30-097 scFvs) and the recombinant antibodies prepared form section (II-2) of the present disclosure (including p30-052, p30-077, p30-090, p30-091, p30-096 and p30-097 IgGs) in structure comprises a VL domain and a VH domain, in which the VL domain comprises CDR-L1, CDR-L2 and CDR-L3, and the VH domain comprises CDR-H1, CDR-H2 and CDR-H3.

According to some embodiments, the CDR-L1, CDR-L2 and CDR-L3 of p30-052 scFv or p30-052 IgG respectively have the amino acid sequences of SEQ ID NOs: 1-3 (i.e., respectively having the amino acid sequences 100% identical to SEQ ID NOs: 1-3), and the CDR-H1, CDR-H2 and CDR-H3 of p30-052 scFv or p30-052 IgG respectively have the amino acid sequences of SEQ ID NOs: 4-6 (i.e., respectively having the amino acid sequences 100% identical to SEQ ID NOs: 4-6). According to some embodiments, the VL domain of p30-052 scFv or p30-052 IgG comprises an amino acid sequence at least 85% (e.g., 85%, 86%, 87%, 88%, 89%, 90%, 91%, 92%, 93%, 94%, 95%, 96%, 97%, 98%, 99% or 100%) identical to SEQ ID NO: 37; and the VH domain of p30-052 scFv or p30-052 IgG comprises an amino acid sequence at least 85% (e.g., 85%, 86%, 87%, 88%, 89%, 90%, 91%, 92%, 93%, 94%, 95%, 96%, 97%, 98%, 99% or 100%) identical to SEQ ID NO: 38. As would be appreciated, the sequence (e.g., the framework sequence) of the VL and VH domains may vary (e.g., being substituted by conserved or non-conserved amino acid residues) without affecting the binding affinity and/or specificity of the present antibody. Preferably, the sequence(s) of the VL and VH domains is/are conservatively substituted by one or more suitable amino acid(s) with similar properties; for example, the substitution of leucine (an nonpolar amino acid residue) by isoleucine, alanine, valine, proline, phenylalanine, or tryptophan (another nonpolar amino acid residue); the substitution of aspartate (an acidic amino acid residue) by glutamate (another acidic amino acid residue); or the substitution of lysine (an basic amino acid residue) by arginine or histidine (another basic amino acid residue). According to the preferred embodiments, the VL and VH domains of p30-052 scFv or p30-052 IgG respectively comprise amino acid sequences at least 90% identical to SEQ ID NOs: 37 and 38. More preferably, the VL and VH domains of p30-052 scFv or p30-052 IgG respectively comprise the amino acid sequences at least 95% identical to SEQ ID NOs: 37 and 38. In one working example of the present disclosure, the VL domain of p30-052 scFv or p30-052 IgG has the amino acid sequence of SEQ ID NO: 37 (i.e., having an amino acid sequence 100% identical to SEQ ID NO: 37), and the VH domain of p30-052 scFv or p30-052 IgG has the amino acid sequence of SEQ ID NO: 38 (i.e., having an amino acid sequence 100% identical to SEQ ID NO: 38).

According to some embodiments, the CDR-L1, CDR-L2 and CDR-L3 of p30-077 scFv or p30-077 IgG respectively have the amino acid sequences of SEQ ID NOs: 7-9, and the CDR-H1, CDR-H2 and CDR-H3 of p30-077 scFv or p30-077 IgG respectively have the amino acid sequences of SEQ ID NOs: 10-12. According to some embodiments, the VL domain of p30-077 scFv or p30-077 IgG comprises an amino acid sequence at least 85% identical to SEQ ID NO: 39; and the VH domain of p30-077 scFv or p30-077 IgG comprises an amino acid sequence at least 85% identical to SEQ ID NO: 40. According to the preferred embodiments, the VL and VH domains of p30-077 scFv or p30-077 IgG respectively comprise the amino acid sequences at least 90% identical to SEQ ID NOs: 39 and 40. More preferably, the VL and VH domains of p30-077 scFv or p30-077 IgG respectively comprise the amino acid sequences at least 95% identical to SEQ ID NOs: 39 and 40. In one working example of the present disclosure, the VL domain of p30-077 scFv or p30-077 IgG has the amino acid sequence of SEQ ID NO: 39, and the VH domain of p30-077 scFv or p30-077 IgG has the amino acid sequence of SEQ ID NO: 40.

According to certain embodiments, the CDR-L1, CDR-L2 and CDR-L3 of p30-090 scFv or p30-090 IgG respectively have the amino acid sequences of SEQ ID NOs: 13-15, and the CDR-H1, CDR-H2 and CDR-H3 of p30-090 scFv or p30-090 IgG respectively have the amino acid sequences of SEQ ID NOs: 16-18. According to some embodiments, the VL domain of p30-090 scFv or p30-090 IgG comprises an amino acid sequence at least 85% identical to SEQ ID NO: 41; and the VH domain of p30-090 scFv or p30-090 IgG comprises an amino acid sequence at least 85% identical to SEQ ID NO: 42. According to the preferred embodiments, the VL and VH domains of p30-090 scFv or p30-090 IgG respectively comprise the amino acid sequences at least 90% identical to SEQ ID NOs: 41 and 42. More preferably, the VL and VH domains of p30-090 scFv or p30-090 IgG respectively comprise the amino acid sequences at least 95% identical to SEQ ID NOs: 41 and 42. In one working example of the present disclosure, the VL domain of p30-090 scFv or p30-090 IgG has the amino acid sequence of SEQ ID NO: 41, and the VH domain of p30-090 scFv or p30-090 IgG has the amino acid sequence of SEQ ID NO: 42.

According to certain embodiments, the CDR-L1, CDR-L2 and CDR-L3 of p30-091 scFv or p30-091 IgG respectively have the amino acid sequences of SEQ ID NOs: 19-21, and the CDR-H1, CDR-H2 and CDR-H3 of p30-091 scFv or p30-091 IgG respectively have the amino acid sequences of SEQ ID NOs: 22-24. According to some embodiments, the VL domain of p30-091 scFv or p30-091 IgG comprises an amino acid sequence at least 85% identical to SEQ ID NO: 43; and the VH domain of p30-091 scFv or p30-091 IgG comprises an amino acid sequence at least 85% identical to SEQ ID NO: 44. According to the preferred embodiments, the VL and VH domains of p30-091 scFv or p30-091 IgG respectively comprise the amino acid sequences at least 90% identical to SEQ ID NOs: 43 and 44. More preferably, the VL and VH domains of p30-091 scFv or p30-091 IgG respectively comprise the amino acid sequences at least 95% identical to SEQ ID NOs: 43 and 44. In one working example of the present disclosure, the VL domain of p30-091 scFv or p30-091 IgG has the amino acid sequence of SEQ ID NO: 43, and the VH domain of p30-091 scFv or p30-091 IgG has the amino acid sequence of SEQ ID NO: 44.

According to some embodiments, the CDR-L1, CDR-L2 and CDR-L3 of p30-096 scFv or p30-096 IgG respectively have the amino acid sequences of SEQ ID NOs: 25-27, and the CDR-H1, CDR-H2 and CDR-H3 of p30-096 scFv or p30-096 IgG respectively have the amino acid sequences of SEQ ID NOs: 28-30. According to some embodiments, the VL domain of p30-096 scFv or p30-096 IgG comprises an amino acid sequence at least 85% identical to SEQ ID NO: 45; and the VH domain of p30-096 scFv or p30-096 IgG comprises an amino acid sequence at least 85% identical to SEQ ID NO: 46. According to the preferred embodiments, the VL and VH domains of p30-096 scFv or p30-096 IgG respectively comprise the amino acid sequences at least 90% identical to SEQ ID NOs: 45 and 46. More preferably, the VL and VH domains of p30-096 scFv or p30-096 IgG respectively comprise the amino acid sequences at least 95% identical to SEQ ID NOs: 45 and 46. In one working example of the present disclosure, the VL domain of p30-096 scFv or p30-096 IgG has the amino acid sequence of SEQ ID NO: 45, and the VH domain of p30-096 scFv or p30-096 IgG has the amino acid sequence of SEQ ID NO: 46.

According to alternative embodiments, the CDR-LI, CDR-L2 and CDR-L3 of p30-097 scFv or p30-097 IgG respectively have the amino acid sequences of SEQ ID NOs: 31-33, and the CDR-H1, CDR-H2 and CDR-H3 of p30-097 scFv or p30-097 IgG respectively have the amino acid sequences of SEQ ID NOs: 34-36. According to some embodiments, the VL domain of p30-097 scFv or p30-097 IgG comprises an amino acid sequence at least 85% identical to SEQ ID NO: 47; and the VH domain of p30-097 scFv or p30-097 IgG comprises an amino acid sequence at least 85% identical to SEQ ID NO: 48. According to the preferred embodiments, the VL and VH domains of p30-097 scFv or p30-097 IgG respectively comprise the amino acid sequences at least 90% identical to SEQ ID NOs: 47 and 48. More preferably, the VL and VH domains of p30-097 scFv or p30-097 IgG respectively comprise the amino acid sequences at least 95% identical to SEQ ID NOs: 47 and 48. In one working example of the present disclosure, the VL domain of p30-097 scFv or p30-097 IgG has the amino acid sequence of SEQ ID NO: 47, and the VH domain of p30-097 scFv or p30-097 IgG has the amino acid sequence of SEQ ID NO: 48.

### (II-4) Uses of Antibody Fragments and Recombinant Antibodies in Diagnosing ASFV Infection

According to some examples of the present disclosure, each of the antibody fragments and recombinant antibodies is useful in detecting ASFV, and accordingly, may serve as a detecting agent for diagnosing ASFV infection.

It is therefore another aspect of the present disclosure to provide a kit for the detection of ASFV infection in a subject (i.e., in a pig). The kit includes, at least, a first antibody, a second antibody, and a container containing the first and second antibodies. Depending on desired purposes, the first and second antibodies are independently selected from the antibody fragments and recombinant antibodies as described in sections (II-1) to (II-3) of the present disclosure. The present kit is useful in detecting the ASFV infection in a biological sample via any detection technique known to a skilled artisan, such as ELISA, LFIA, SPR, western blotting assay, and flow cytometry. According to some working examples, the first and second antibody fragments/recombinant antibodies of the present kit respectively serve as a capture antibody and a detection antibody for use in ELISA, western blotting assay or LFIA.

In one example, the kit designated as “D96C77” comprises p30-077 IgG as the first antibody, and p30-096 IgG as the second antibody. In another example, the kit designated as “D91C90” comprises p30-090 IgG as the first antibody, and p30-091 IgG as the second antibody. In still another example, the kit designated as “D97C52” comprises p30-052 IgG as the first antibody, and p30-097 IgG as the second antibody.

Optionally, the kit may further comprise a legend indicating how to use the antibody fragment or the recombinant antibody for detecting ASFV infection.

Also included herein is a method of making a diagnosis of whether a subject is infected by ASFV via a biological sample isolated from the subject. The method comprises detecting the presence or absence of a viral protein of the ASFV in the biological sample by use of the antibody fragment, the recombinant antibody, or the kit of the present disclosure, wherein when the viral protein (e.g., p30 protein) is present in the biological sample, then diagnosing that the subject is infected by the ASFV.

Non-limiting examples of the biological sample suitable to be used in the present method include, whole blood, serum, plasma, kidney, liver, lung, spleen, lymph node, spleen, tonsil and small intestine of the subject. Alternatively, the biological sample may be an oral, nasal, or anal swab sample of the subject.

Based on the diagnostic result, a skilled artisan or a clinical practitioner may administer to a subject need thereof (i.e., an ASFV-infected swine) an effective amount of an anti-viral treatment thereby ameliorating and/or alleviating the symptom(s) associated with the ASFV infection. Examples of the anti-viral treatment suitable to be used in the present method include, but are not limited to, apigenin, enrofloxacin, grepafloxacin, balofloxacin, tosufloxacin, gatifloxacin, garenoxacin, or a combination thereof. Alternatively, the subject diagnosed as having ASFV infection (i.e., the ASFV-infected swine) is promptly isolated from other livestock on the farm so as to prevent and control the spread of ASFV.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### Cell Lines

Suspension Expi293F™ cells were cultured in Expi293™ Expression Medium at 37° C. with shaking 110 rpm in 8% CO₂ incubator.

Characterization of the IgG1s Derived from the Selection and Screening Procedure with Phage-Displayed Synthetic scFv Libraries

The construction and characterization of the phage-displayed synthetic scFv libraries followed the same procedure, without modification, as described in the co-pending PCT applications, PCT/US2016/19128 and PCT/US2018/56627. The experimental procedures for panning the phage display libraries, selecting and screening of phage-displayed scFv binders, characterizing the scFvs binding to the cognate antigens and Protein A/L with ELISA, reformatting scFvs into IgG1s, and expressing and purifying IgG1s have been described in the co-pending PCT applications, PCT/US2016/19128 and PCT/US2018/56627.

Detection of p30 Protein with Immunoblotting

The recombinant p30 protein (250 ng) was resolved in 12% sodium dodecyl sulfate polyacrylamide gel electrophoresis (SDS-PAGE), followed by transferring to a polyvinylidene difluoride nylon (PVDF) membrane, and probed with specified IgG antibodies (1 ug/mL). The specific bands were detected by HRP-conjugated antibody (1 mg/ml, 1:2,500 dilution), and revealed by chemiluminescent substrate and chemiluminescent imaging system.

### EC₅₀ for Antibody-Antigen Interaction

The IgG EC₅₀ was determined by the titrations of IgG antibody on immobilized recombinant p30 protein with ELISA. Specifically, the p30 protein (0.5 μg per well) diluted in PBS buffer (pH 7.4) was coated on 96-well immunoplates, and then blocked with 5% skim milk in PBST for 1 hour. In the meantime, two-fold serial dilutions of the antibody (stock concentration: 0.5 μg/mL) in PBST with 5% milk were performed, and 11 different concentrations of the antibody were generated. After blocking, 100 μL diluted antibody samples were added to each well, and incubated for 1 hour under gentle shaking. The wells were washed three times with 300 μL PBST, and then reacted with 100 μL of 1:5000-diluted HRP-conjugated goat anti-human IgG antibody in PBST with 5% milk for 1 hour incubation. The wells were washed three times with 300 μL PBST buffer and twice with 300 μL PBS, developed for 5 minutes with TMB substrate, quenched with 1.0 M HCl and read spectrophotometrically at 450 nm.

Detection of p30 Protein with Sandwich ELISA

Capture antibody (1 μg per well) diluted in PBS buffer (pH7.4) was coated on 96-well immunoplates overnight. The plates were blocked with 5% skim milk in PBST for 1 hour. Then, two-fold serial dilutions of p30 protein (stock concentration: 1 μg/mL) were added to the plates (100 μL per well), and incubated at room temperature for 1 hour under gentle shaking. The wells were washed three times with 300 μL PBST, and then reacted with 100 μL HRP-conjugated detection antibody (1:2000 dilution) in 5% milk/PBST for 1 hour incubation with gently shaking at room temperature. The wells were washed three times with 300 μL PBST buffer and twice with 300 μL PBS, developed for 5 minutes with TMB substrate, quenched with 1.0 M HCl and read spectrophotometrically at 450 nm.

### Preparation of Colloidal Gold-Conjugated AL2C and IgGs

50 μl of 0.2 M K₂CO₃ (pH 11.5) was mixed with 10 ml colloidal gold solution (pH 5-6) to adjust pH (final pH 8), and then added 500 μg of IgG or 167 μg of AL2C to the colloidal gold solution to react 40 minutes at room temperature. The reaction was stopped by adding 1 ml of blocking buffer (10% bovine serum albumin (BSA) in 20 mM sodium borate, pH 9.3) for 15 minutes at room temperature, followed by centrifugation (15,000 xg, 30 minutes, 4° C.). The supernatant was discarded, and the pellet was completely resuspended in 10 ml wash buffer (1% BSA in 20 mM sodium borate, pH 9.3), followed by centrifugation (15,000 xg, 30 minutes, 4° C.). The washing procedure was repeated twice, and the pellet was resuspended in 1 ml of 1% BSA in 20 mM sodium borate (pH 9.3) for the procedure preparing the conjugate pad.

### Assembly of the LFIA Strips

1 μg of the capture antibody, antigen or AL2C in PBS buffer were stripped on nitrocellulose membrane per cm with lateral flow dispenser driven by syringe infusion pump. All other procedures for the preparation of the nitrocellulose membrane with immobilized antigen or capture antibody, the preparation of the conjugate pad and the sample pad, and the preparation of the LFIA strip assembly were followed the protocol previously reported.

### Example 1 Characterization of Recombinant Antibodies

The p30 protein of ASFV was synthesized for selecting a panel of anti-p30 antibodies in accordance with the methodology described in the co-pending PCT applications, PCT/US2016/19128 and PCT/US2018/56627.

6 anti-p30 scFvs were selected from the phage-displayed scFv libraries, and reformatted into IgG1s, which were respectively designated as “p30-052 IgG”, “p30-077 IgG”, “p30-090 IgG”, “p30-091 IgG”, “p30-096 IgG” and “p30-097 IgG”. The VL and VH sequences of these antibodies were summarized in Table 1. According to the analytic result of ELISA, the binding affinities (EC₅₀) of p30-077, p30-096, p30-090, p30-091, p30-052 and p30-097 IgGs to the p30 protein were respectively 2.6, 2.5, 0.9, 2.6, 2, and 1.2 ng/ml.

TABLE 1
VL and VH sequences of specified antibodies

| | | | |
|---|---|---|---|
| | | | SEQ |
| | | | ID |
| Name | Domain | Amino acid sequence | NO |

p30

| | | | |
|---|---|---|---|
| p30-052 | VL | MADIQMTQSPSSLSASVGDRVTITCRASQDVDNNVAWYQQKPGKAP | 37 |
| | | KLLISSPGFLYSGVPSRFSGSGSGTDFTLTISSLQPEDFATYYCQQ | |
| | | YFNFPITFGQGTKVEIKR | |
| | VH | EVQLVESGGGLVQPGGSLRLSCAASGFTIDSGFIHWVRQAPGKGLEW | 38 |
| | | VAGIGPFWGFTFYADSVKGRFTISADTSKNTAYLQMNSLRAEDTAV | |
| | | YYCARYGSSSYGFDYWGQGTLVTVSSASAAA | |
| p30-077 | VL | MADIQMTQSPSSLSASVGDRVTITCRASQDVNNNVAWYQQKPGKAP | 39 |
| | | KLLISSPTYLYSGVPSRFSGSGSGTDFTLTISSLQPEDFATYYCQQ | |
| | | WYDWPITFGQGTKVEIKR | |
| | VH | EVQLVESGGGLVQPGGSLRLSCAASGFTIGDWGIHWVRQAPGKGLE | 40 |
| | | WVAGIWPFGGFTFYADSVKGRFTISADTSKNTAYLQMNSLRAEDTA | |
| | | VYYCARGSFYWDYWGQGTLVTVSSASAAA | |
| p30-090 | VL | MADIQMTQSPSSLSASVGDRVTITCRASQDVNNDVAWYQQKPGKAP | 41 |
| | | KLLIFYPGGLYSGVPSRFSGSGSGTDFTLTISSLQPEDFATYYCQQ | |
| | | SWNYPLTFGQGTKVEIKR | |
| | VH | EVQLVESGGGLVQPGGSLRLSCAASGFTIDGGGIHWVRQAPGKGLE | 42 |
| | | WVAGIWPSWGYTYYADSVKGRFTISADTSKNTAYLQMNSLRAEDTA | |
| | | VYYCARGFYWYDYWGQGTLVTVSSASAAA | |
| p30-091 | VL | MADIQMTQSPSSLSASVGDRVTITCKSNQNLLDSGDQGTYLNWYQQ | 43 |
| | | KPGKAPKLLISDVSNLYSGVPSRFSGSGSGTDFTLTISSLQPEDFA | |
| | | TYYCYQATSFPFTFGQGTKVEIKR | |
| | VH | EVQLVESGGGLVQPGGSLRLSCAASGFTISDYWIHWVRQAPGKGLE | 44 |
| | | WVAYINPHYGLTDYADSVKGRFTISADTSKNTAYLQMNSLRAEDTA | |
| | | VYYCARGWLLDYWGQGTLVTVSSASAAA | |
| p30-096 | VL | MADIQMTQSPSSLSASVGDRVTITCSGSSSNIGDNDVYWYWYQQKPG | 45 |
| | | KAPKLLIYGTTSLYSGVPSRFSGSGSGTDFTLTISSLQPEDFATYYC | |
| | | AAGGWGHNGVFGQGTKVEIKR | |
| | VH | EVQLVESGGGLVQPGGSLRLSCAASGFTISDWSIHWVWVRQAPGKG | 46 |
| | | LEWVAGIWPYWGFTSYADSVKGRFTISADTSKNTAYLQMNSLRAED | |
| | | TAVYYCARFVSYYGYSLMDYWGQGTLVTVSSASAAA | |
| p30-097 | VL | MADIQMTQSPSSLSASVGDRVTITCKSNQNLLHSDGGTSLHWYWYQ | 47 |
| | | QKPGKAPKLLIFDTSNLYSGVPSRFSGSGSGTDFTLTISSLQPEDF | |
| | | ATYYCYQTSRLPGFGQGTKVEIKR | |
| | VH | EVQLVESGGGLVQPGGSLRLSCKASGYTFTTFWMYWVWVRQAPGK | 48 |
| | | GLEWVAGIDPHHGLTYYADSVKGRFTISADTSKNTAYLQMNSLRAE | |
| | | DTAVYYCARGDWDGLMDYWGQGTLVTVSSASAAA | |

* The CDR sequences were marked in boldface, including three CDRs (i.e., CDR-L1, CDR-L2 and CDR-L3, from N-terminus to C-terminus, in sequence) in the VL domain, and three CDRs (i.e., CDR-H1, CDR-H2 and CDR-H3, from N-terminus to C-terminus, in sequence) in the VH domain.

The binding affinities of p30-077, p30-096, p30-090, p30-091, p30-052 and p30-097 IgGs to the p30 protein of ASFV were further examined by sandwich ELISA. As the data summarized in Table 2, the present antibodies were useful in serving as the detection and capture antibodies for detecting ASFV, in which the EC₅₀ of antibody pair D96C77 against ASFV was 16.52 ng/ml; the EC₅₀ of antibody pair D91C90 was 8.91 ng/ml; and the EC₅₀ of antibody pair D97C52 was 8.43 ng/mL.

TABLE 2
Sandwich ELISA EC₅₀ of specified antibodies
against the p30 protein of ASFV

| Antibody | EC₅₀ | |
|---|---|---|
| pair | (ng/ml) | Note |
| D96C77 | 16.52 | Detection antibody: p30-096 IgG |
| | | Capture antibody: p30-077 IgG |
| D91C90 | 8.91 | Detection antibody: p30-091 IgG |
| | | Capture antibody: p30-090 IgG |
| D97C52 | 8.43 | Detection antibody: p30-097 IgG |
| | | Capture antibody: p30-052 IgG |

In addition to ELISA, the present antibodies may also serve as detecting antibodies for ASFV in western blotting assay. According to the result, p30-007, p30-011, p30-014, p30-015, p30-019 and p30-020 IgGs were capable of specifically recognizing the p30 protein without binding to the p54 protein (another structural protein of ASFV) (data not shown).

### Example 2 Establishing LFIA Devices for Detecting ASFV

For the purpose of constructing LFIA devices, 6 anti-p30 antibodies were conjugated to colloidal gold in accordance with the procedures described in “Materials and Methods” of the present disclosure. The analytic results indicated that the present antibodies may efficiently conjugate to colloidal gold (data not shown).

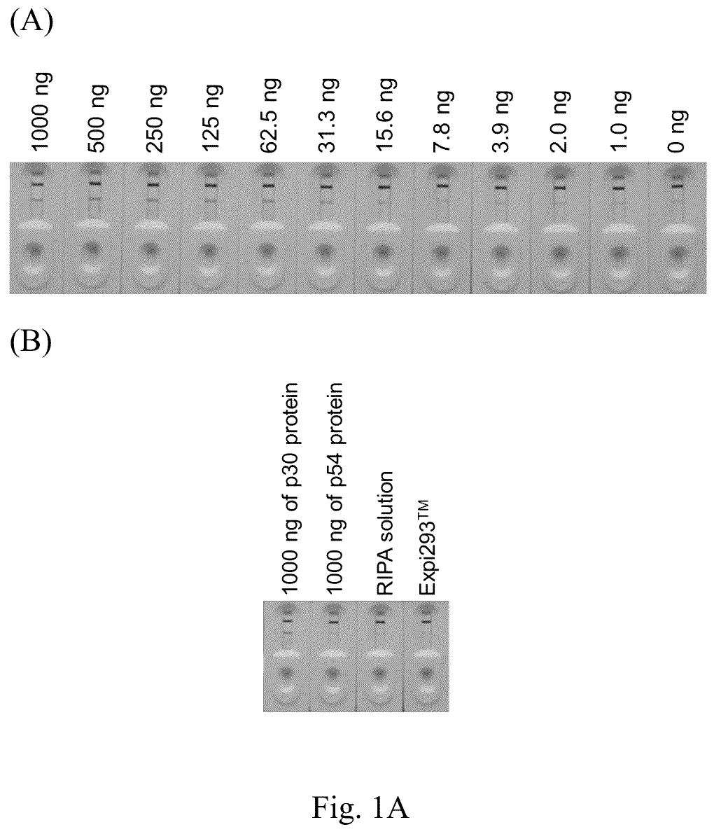

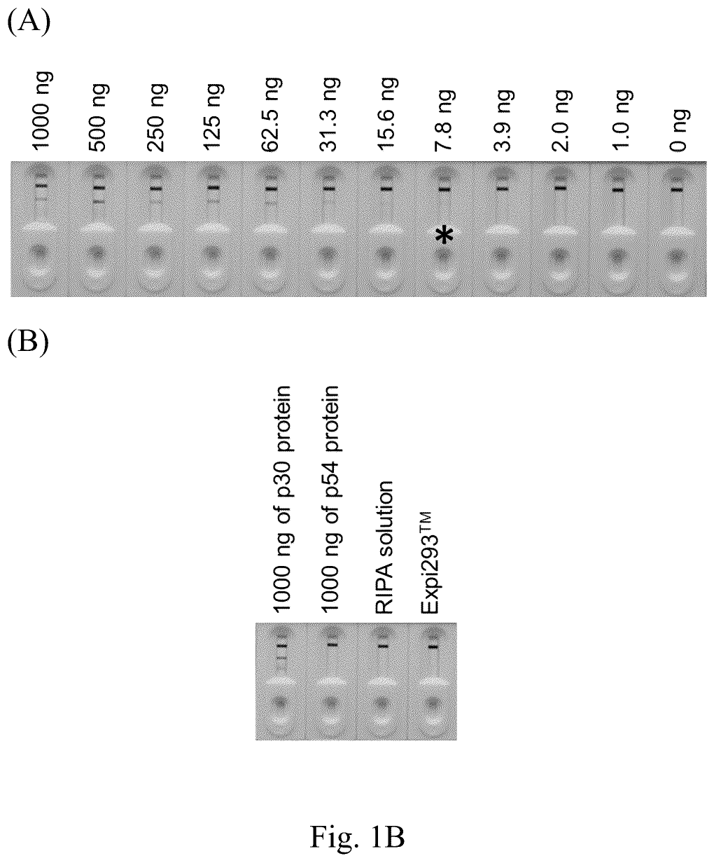

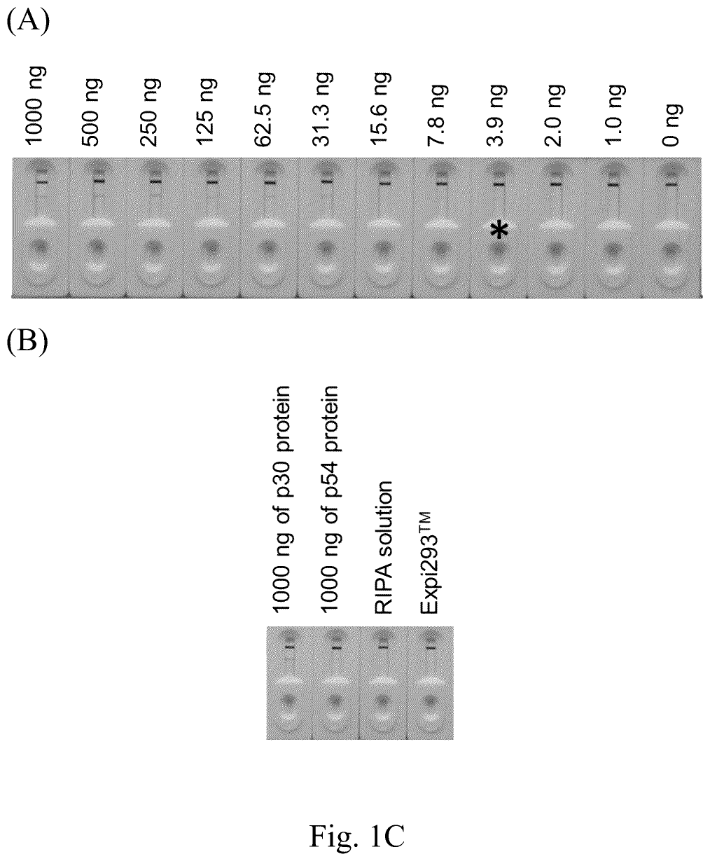

Three prototypes of LFIA device were established in this example, in which the D96C77 LFIA prototype was constructed with p30-077 IgG as the capture antibody and p30-096 IgG as the colloidal gold-conjugated detection antibody, the D91C90 LFIA prototype was constructed with p30-090 IgG as the capture antibody and p30-091 IgG as the colloidal gold-conjugated detection antibody, and the D97C52 LFIA prototype was constructed with p30-052 IgG as the capture antibody and p30-097 IgG as the colloidal gold-conjugated detection antibody. The detection limit of each LFIA prototype was determined with two samples containing the p30 protein of ASFV. The first sample contained Expi293™ cell-expressed p30 protein, and the second sample contained E. coli-expressed p30 protein. According to the data of Panel (A) of FIGS. 1A to 1C, each of the antibody pair D96C77 (FIG. 1A), antibody pair D91C90 (FIG. 1B) and antibody pair D97C52 (FIG. 1C) was capable of binding to the Expi293™ cell-expressed p30 protein, and the detection limits were in the range of 1.0-7.8 ng/test. The data of Panel (B) of FIGS. 1A to 1C further demonstrated that each of the antibody pairs exhibited a binding specificity to the Expi293™ cell-expressed p30 protein.

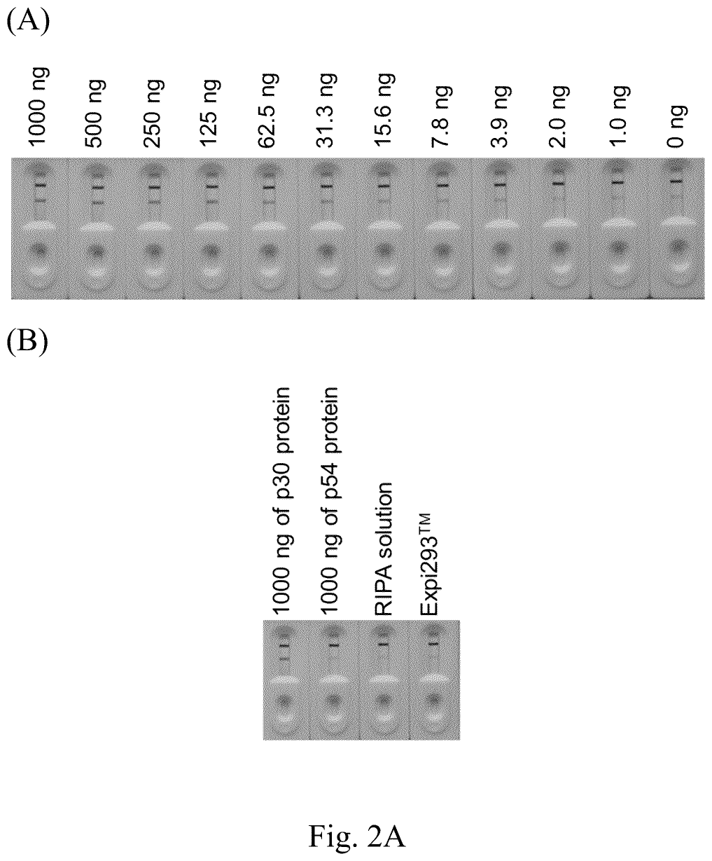

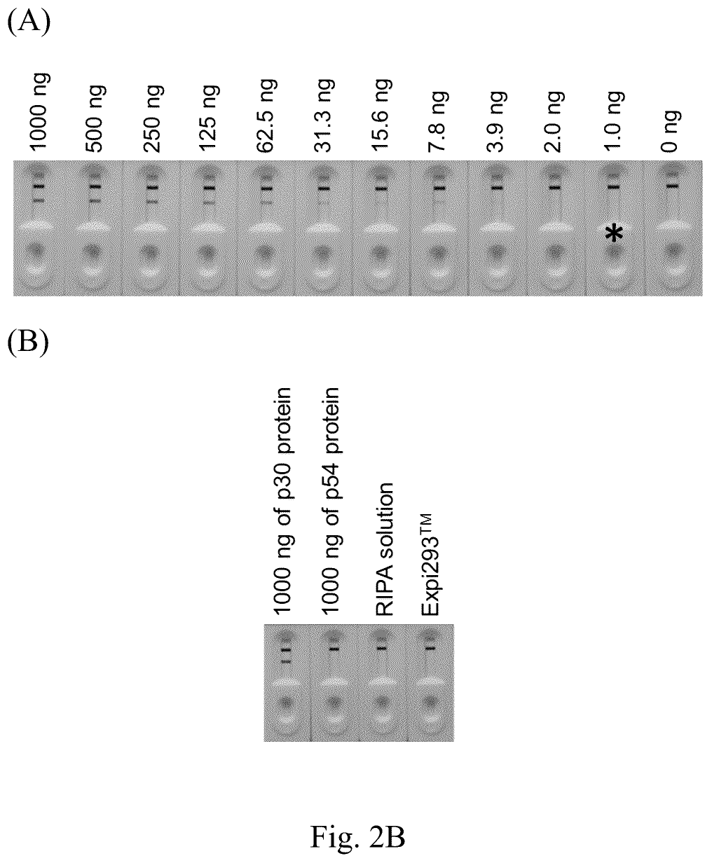

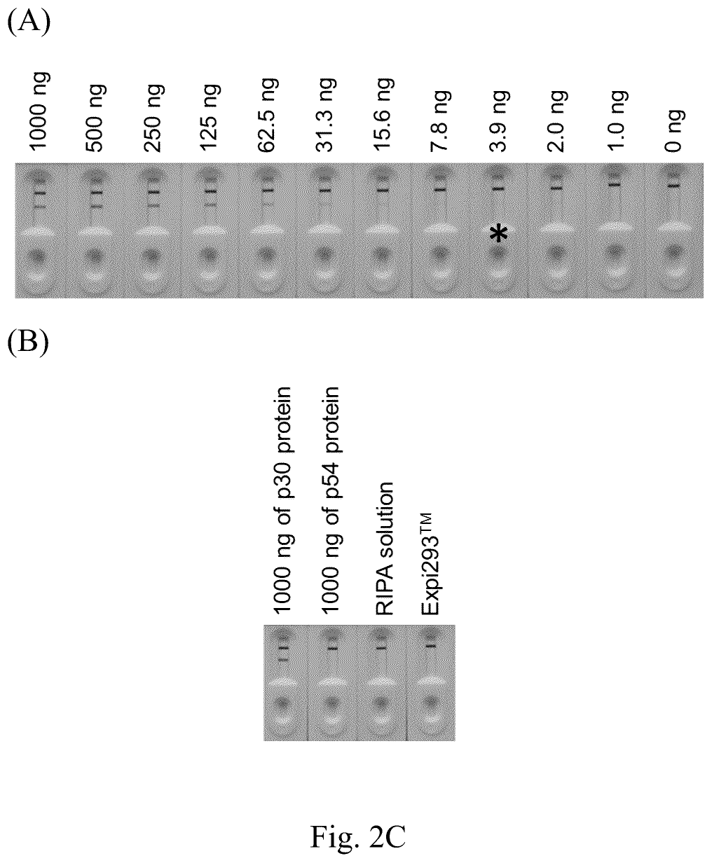

According to the data of Panel (A) of FIGS. 2A to 2C, each of the antibody pair D96C77 (FIG. 2A), antibody pair D91C90 (FIG. 2B) and antibody pair D97C52 (FIG. 2C) was capable of binding to the E. coli-expressed p30 protein, and the detection limits were in the range of 1.0-3.9 ng/test. The data of Panel (B) of FIGS. 2A to 2C also confirmed that each of the antibody pairs exhibited a binding specificity to the E. coli-expressed p30 protein.

In summary, the antibodies selected from the GH synthetic antibody libraries were demonstrated to be capable of binding to p30 protein of ASFV with high affinities and specificities. The present antibodies (including 6 anti-p30 IgGs) derived from the GH antibody libraries without further affinity maturation were used in sandwich ELISA and LFIA to detect the corresponding viral proteins with detection limit of 1.0 ng/test. Accordingly, the present antibodies may serve as potential antibodies for detecting ASFV thereby preventing and controlling the spread of the infectious disease.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A recombinant antibody or the fragment thereof, comprising a variable light chain (VL) domain and a variable heavy chain (VH) domain, wherein
the VL domain comprises the amino acid sequences of SEQ ID NOs: 1-3, and the VH domain comprises the amino acid sequences of SEQ ID NOs: 4-6;
the VL domain comprises the amino acid sequences of SEQ ID NOs: 7-9, and the VH domain comprises the amino acid sequences of SEQ ID NOS: 10-12;
the VL domain comprises the amino acid sequences of SEQ ID NOS: 13-15, and the VH domain comprises the amino acid sequences of SEQ ID NOs: 16-18;
the VL domain comprises the amino acid sequences of SEQ ID NOS: 19-21, and the VH domain comprises the amino acid sequences of SEQ ID NOs: 22-24;
the VL domain comprises the amino acid sequences of SEQ ID NOS: 25-27, and the VH domain comprises the amino acid sequences of SEQ ID NOs: 28-30; or
the VL domain comprises the amino acid sequences of SEQ ID NOs: 31-33, and the VH domain comprises the amino acid sequences of SEQ ID NOs: 34-36.

2. The recombinant antibody of claim 1, wherein
the VL domain comprises an amino acid sequence at least 85% identical to SEQ ID NO: 37, and the VH domain comprises an amino acid sequence at least 85% identical to SEQ ID NO: 38;
the VL domain comprises an amino acid sequence at least 85% identical to SEQ ID NO: 39, and the VH domain comprises an amino acid sequence at least 85% identical to SEQ ID NO: 40;
the VL domain comprises an amino acid sequence at least 85% identical to SEQ ID NO: 41, and the VH domain comprises an amino acid sequence at least 85% identical to SEQ ID NO: 42;
the VL domain comprises an amino acid sequence at least 85% identical to SEQ ID NO: 43, and the VH domain comprises an amino acid sequence at least 85% identical to SEQ ID NO: 44;
the VL domain comprises an amino acid sequence at least 85% identical to SEQ ID NO: 45, and the VH domain comprises an amino acid sequence at least 85% identical to SEQ ID NO: 46; or
the VL domain comprises an amino acid sequence at least 85% identical to SEQ ID NO: 47, and the VH domain comprises an amino acid sequence at least 85% identical to SEQ ID NO: 48.

3. The recombinant antibody of claim 2, wherein
the VL domain comprises an amino acid sequence 100% identical to SEQ ID NO: 37, and the VH domain comprises an amino acid sequence 100% identical to SEQ ID NO: 38;
the VL domain comprises an amino acid sequence 100% identical to SEQ ID NO: 39, and the VH domain comprises an amino acid sequence 100% identical to SEQ ID NO: 40;
the VL domain comprises an amino acid sequence 100% identical to SEQ ID NO: 41, and the VH domain comprises an amino acid sequence 100% identical to SEQ ID NO: 42;
the VL domain comprises an amino acid sequence 100% identical to SEQ ID NO: 43, and the VH domain comprises an amino acid sequence 100% identical to SEQ ID NO: 44;
the VL domain comprises an amino acid sequence 100% identical to SEQ ID NO: 45, and the VH domain comprises an amino acid sequence 100% identical to SEQ ID NO: 46; or
the VL domain comprises an amino acid sequence 100% identical to SEQ ID NO: 47, and the VH domain comprises an amino acid sequence 100% identical to SEQ ID NO: 48.

4. A kit for detecting African swine fever virus (ASFV), comprising a first recombinant antibody and a second recombinant antibody, independently according to any of claim 1; and a container containing the first and second recombinant antibodies.

5. The kit of claim 4, wherein
the VL domain of the first recombinant antibody comprises the amino acid sequences of SEQ ID NOs: 1-3, and the VH domain of the first recombinant antibody comprises the amino acid sequences of SEQ ID NOs: 4-6; and
the VL domain of the second recombinant antibody comprises the amino acid sequences of SEQ ID NOs: 31-33, and the VH domain of the second recombinant antibody comprises the amino acid sequences of SEQ ID NOs: 34-36.

6. The kit of claim 5, wherein
the VL domain of the first recombinant antibody comprises an amino acid sequence at least 85% identical to SEQ ID NO: 37, and the VH domain of the first recombinant antibody comprises an amino acid sequence at least 85% identical to SEQ ID NO: 38; and
the VL domain of the second recombinant antibody comprises an amino acid sequence at least 85% identical to SEQ ID NO: 47, and the VH domain of the second recombinant antibody comprises an amino acid sequence at least 85% identical to SEQ ID NO: 48.

7. The kit of claim 6, wherein
the VL domain of the first recombinant antibody comprises an amino acid sequence 100% identical to SEQ ID NO: 37, and the VH domain of the first recombinant antibody comprises an amino acid sequence 100% identical to SEQ ID NO: 38; and
the VL domain of the second recombinant antibody comprises an amino acid sequence 100% identical to SEQ ID NO: 47, and the VH domain of the second recombinant antibody comprises an amino acid sequence 100% identical to SEQ ID NO: 48.

8. The kit of claim 4, wherein
the VL domain of the first recombinant antibody comprises the amino acid sequences of SEQ ID NOs: 7-9, and the VH domain of the first recombinant antibody comprises the amino acid sequences of SEQ ID NOs: 10-12; and
the VL domain of the second recombinant antibody comprises the amino acid sequences of SEQ ID NOs: 25-27, and the VH domain of the second recombinant antibody comprises the amino acid sequences of SEQ ID NOS: 28-30.

9. The kit of claim 8, wherein
the VL domain of the first recombinant antibody comprises an amino acid sequence at least 85% identical to SEQ ID NO: 39, and the VH domain of the first recombinant antibody comprises an amino acid sequence at least 85% identical to SEQ ID NO: 40; and
the VL domain of the second recombinant antibody comprises an amino acid sequence at least 85% identical to SEQ ID NO: 45, and the VH domain of the second recombinant antibody comprises an amino acid sequence at least 85% identical to SEQ ID NO: 46.

10. The kit of claim 9, wherein
the VL domain of the first recombinant antibody comprises an amino acid sequence 100% identical to SEQ ID NO: 39, and the VH domain of the first recombinant antibody comprises an amino acid sequence 100% identical to SEQ ID NO: 40; and
the VL domain of the second recombinant antibody comprises an amino acid sequence 100% identical to SEQ ID NO: 45, and the VH domain of the second recombinant antibody comprises an amino acid sequence 100% identical to SEQ ID NO: 46.

11. The kit of claim 4, wherein
the VL domain of the first recombinant antibody comprises the amino acid sequences of SEQ ID NOs: 13-15, and the VH domain of the first recombinant antibody comprises the amino acid sequences of SEQ ID NOs: 16-18; and
the VL domain of the second recombinant antibody comprises the amino acid sequences of SEQ ID NOs: 19-21, and the VH domain of the second recombinant antibody comprises the amino acid sequences of SEQ ID NOs: 22-24.

12. The kit of claim 11, wherein
the VL domain of the first recombinant antibody comprises an amino acid sequence at least 85% identical to SEQ ID NO: 41, and the VH domain of the first recombinant antibody comprises an amino acid sequence at least 85% identical to SEQ ID NO: 42; and
the VL domain of the second recombinant antibody comprises an amino acid sequence at least 85% identical to SEQ ID NO: 43, and the VH domain of the second recombinant antibody comprises an amino acid sequence at least 85% identical to SEQ ID NO: 44.

13. The kit of claim 12, wherein
the VL domain of the first recombinant antibody comprises an amino acid sequence 100% identical to SEQ ID NO: 41, and the VH domain of the first recombinant antibody comprises an amino acid sequence 100% identical to SEQ ID NO: 42; and
the VL domain of the second recombinant antibody comprises an amino acid sequence 100% identical to SEQ ID NO: 43, and the VH domain of the second recombinant antibody comprises an amino acid sequence 100% identical to SEQ ID NO: 44.

14. A method of determining whether a subject is infected by ASFV via a biological sample isolated from the subject, comprising detecting the presence or absence of a viral protein of the ASFV in the biological sample by use of the kit of claim 4, wherein when the presence of the viral protein indicates that the subject is infected by the ASFV.

15. The method of claim 14, wherein the viral protein is 30-kDa phosphoprotein.


SEQUENCE LISTING (53 sequences)

SEQ ID NO: 1 - protein, 11 aa, Artificial Sequence
  synthesized_p30-052_CDR-L1
RASQDVDNNVA

SEQ ID NO: 2 - protein, 8 aa, Artificial Sequence
  synthesized_p30-052_CDR-L2
SSPGFLYS

SEQ ID NO: 3 - protein, 9 aa, Artificial Sequence
  synthesized_p30-052_CDR-L3
QQYFNFPIT

SEQ ID NO: 4 - protein, 13 aa, Artificial Sequence
  synthesized_p30-052_CDR-H1
AASGFTIDSGFIH

SEQ ID NO: 5 - protein, 10 aa, Artificial Sequence
  synthesized_p30-052_CDR-H2
GIGPFWGFTF

SEQ ID NO: 6 - protein, 12 aa, Artificial Sequence
  synthesized__p30-052_CDR-H3
ARYGSSSYGFDY

SEQ ID NO: 7 - protein, 11 aa, Artificial Sequence
  synthesized_p30-077_CDR-L1
RASQDVNNNVA

SEQ ID NO: 8 - protein, 8 aa, Artificial Sequence
  synthesized_p30-077_CDR-L2
SSPTYLYS

SEQ ID NO: 9 - protein, 9 aa, Artificial Sequence
  synthesized_p30-077_CDR-L3
QQWYDWPIT

SEQ ID NO: 10 - protein, 13 aa, Artificial Sequence
  synthesized_p30-077_CDR-H1
AASGFTIGDWGIH

SEQ ID NO: 11 - protein, 10 aa, Artificial Sequence
  synthesized_p30-077_CDR-H2
GIWPFGGFTF

SEQ ID NO: 12 - protein, 9 aa, Artificial Sequence
  synthesized_p30-077_CDR-H3
ARGSFYWDY

SEQ ID NO: 13 - protein, 11 aa, Artificial Sequence
  synthesized_p30-090_CDR-L1
RASQDVNNDVA

SEQ ID NO: 14 - protein, 8 aa, Artificial Sequence
  synthesized_p30-090_CDR-L2
FYPGGLYS

SEQ ID NO: 15 - protein, 9 aa, Artificial Sequence
  synthesized_p30-090_CDR-L3
QQSWNYPLT

SEQ ID NO: 16 - protein, 13 aa, Artificial Sequence
  synthesized_p30-090_CDR-H1
AASGFTIDGGGIH

SEQ ID NO: 17 - protein, 10 aa, Artificial Sequence
  synthesized_p30-090_CDR-H2
GIWPSWGYTY

SEQ ID NO: 18 - protein, 9 aa, Artificial Sequence
  synthesized_p30-090_CDR-H3
ARGFYWYDY

SEQ ID NO: 19 - protein, 17 aa, Artificial Sequence
  synthesized_p30-091_CDR-L1
KSNQNLLDSGDQGTYLN

SEQ ID NO: 20 - protein, 8 aa, Artificial Sequence
  synthesized_p30-091_CDR-L2
SDVSNLYS

SEQ ID NO: 21 - protein, 9 aa, Artificial Sequence
  synthesized_p30-091_CDR-L3
YQATSFPFT

SEQ ID NO: 22 - protein, 13 aa, Artificial Sequence
  synthesized_p30-091_CDR-H1
AASGFTISDYWIH

SEQ ID NO: 23 - protein, 10 aa, Artificial Sequence
  synthesized_p30-091_CDR-H2
YINPHYGLTD

SEQ ID NO: 24 - protein, 8 aa, Artificial Sequence
  synthesized_p30-091_CDR-H3
ARGWLLDY

SEQ ID NO: 25 - protein, 15 aa, Artificial Sequence
  synthesized_p30-096_CDR-L1
SGSSSNIGDNDVYWY

SEQ ID NO: 26 - protein, 8 aa, Artificial Sequence
  synthesized_p30-096_CDR-L2
YGTTSLYS

SEQ ID NO: 27 - protein, 10 aa, Artificial Sequence
  synthesized_p30-096_CDR-L3
AAGGWGHNGV

SEQ ID NO: 28 - protein, 15 aa, Artificial Sequence
  synthesized_p30-096_CDR-H1
AASGFTISDWSIHWV

SEQ ID NO: 29 - protein, 10 aa, Artificial Sequence
  synthesized_p30-096_CDR-H2
GIWPYWGFTS

SEQ ID NO: 30 - protein, 14 aa, Artificial Sequence
  synthesized_p30-096_CDR-H3
ARFVSYYGYSLMDY

SEQ ID NO: 31 - protein, 18 aa, Artificial Sequence
  synthesized_p30-097_CDR-L1
KSNQNLLHSDGGTSLHWY

SEQ ID NO: 32 - protein, 8 aa, Artificial Sequence
  synthesized_p30-097_CDR-L2
FDTSNLYS

SEQ ID NO: 33 - protein, 8 aa, Artificial Sequence
  synthesized_p30-097_CDR-L3
YQTSRLPG

SEQ ID NO: 34 - protein, 15 aa, Artificial Sequence
  synthesized_p30-097_CDR-H1
KASGYTFTTFWMYWV

SEQ ID NO: 35 - protein, 10 aa, Artificial Sequence
  synthesized_p30-097_CDR-H2
GIDPHHGLTY

SEQ ID NO: 36 - protein, 11 aa, Artificial Sequence
  synthesized_p30-097_CDR-H3
ARGDWDGLMDY

SEQ ID NO: 37 - protein, 110 aa, Artificial Sequence
  synthesized_p30-052_VL
MADIQMTQSPSSLSASVGDRVTITCRASQDVDNNVAWYQQKPGKAPKLLISSPGFLYSGVPSRFSGSGSGTDFTLTISSLQPEDFATYYCQQYFNFPITFGQGTKVEIKR

SEQ ID NO: 38 - protein, 124 aa, Artificial Sequence
  synthesized_p30-052_VH
EVQLVESGGGLVQPGGSLRLSCAASGFTIDSGFIHWVRQAPGKGLEWVAGIGPFWGFTFYADSVKGRFTISADTSKNTAYLQMNSLRAEDTAVYYCARYGSSSYGFDYWGQGTLVTVSSASAAA

SEQ ID NO: 39 - protein, 110 aa, Artificial Sequence
  synthesized_p30-077_VL
MADIQMTQSPSSLSASVGDRVTITCRASQDVNNNVAWYQQKPGKAPKLLISSPTYLYSGVPSRFSGSGSGTDFTLTISSLQPEDFATYYCQQWYDWPITFGQGTKVEIKR

SEQ ID NO: 40 - protein, 121 aa, Artificial Sequence
  synthesized_p30-077_VH
EVQLVESGGGLVQPGGSLRLSCAASGFTIGDWGIHWVRQAPGKGLEWVAGIWPFGGFTFYADSVKGRFTISADTSKNTAYLQMNSLRAEDTAVYYCARGSFYWDYWGQGTLVTVSSASAAA

SEQ ID NO: 41 - protein, 110 aa, Artificial Sequence
  synthesized_p30-090_VL
MADIQMTQSPSSLSASVGDRVTITCRASQDVNNDVAWYQQKPGKAPKLLIFYPGGLYSGVPSRFSGSGSGTDFTLTISSLQPEDFATYYCQQSWNYPLTFGQGTKVEIKR

SEQ ID NO: 42 - protein, 121 aa, Artificial Sequence
  synthesized_p30-090_VH
EVQLVESGGGLVQPGGSLRLSCAASGFTIDGGGIHWVRQAPGKGLEWVAGIWPSWGYTYYADSVKGRFTISADTSKNTAYLQMNSLRAEDTAVYYCARGFYWYDYWGQGTLVTVSSASAAA

SEQ ID NO: 43 - protein, 116 aa, Artificial Sequence
  synthesized_p30-091_VL
MADIQMTQSPSSLSASVGDRVTITCKSNQNLLDSGDQGTYLNWYQQKPGKAPKLLISDVSNLYSGVPSRFSGSGSGTDFTLTISSLQPEDFATYYCYQATSFPFTFGQGTKVEIKR

SEQ ID NO: 44 - protein, 120 aa, Artificial Sequence
  synthesized_p30-091_VH
EVQLVESGGGLVQPGGSLRLSCAASGFTISDYWIHWVRQAPGKGLEWVAYINPHYGLTDYADSVKGRFTISADTSKNTAYLQMNSLRAEDTAVYYCARGWLLDYWGQGTLVTVSSASAAA

SEQ ID NO: 45 - protein, 115 aa, Artificial Sequence
  synthesized_p30-096_VL
MADIQMTQSPSSLSASVGDRVTITCSGSSSNIGDNDVYWYWYQQKPGKAPKLLIYGTTSLYSGVPSRFSGSGSGTDFTLTISSLQPEDFATYYCAAGGWGHNGVFGQGTKVEIKR

SEQ ID NO: 46 - protein, 128 aa, Artificial Sequence
  synthesized_p30-096_VH
EVQLVESGGGLVQPGGSLRLSCAASGFTISDWSIHWVWVRQAPGKGLEWVAGIWPYWGFTSYADSVKGRFTISADTSKNTAYLQMNSLRAEDTAVYYCARFVSYYGYSLMDYWGQGTLVTVSSASAAA

SEQ ID NO: 47 - protein, 116 aa, Artificial Sequence
  synthesized_p30-097_VL
MADIQMTQSPSSLSASVGDRVTITCKSNQNLLHSDGGTSLHWYWYQQKPGKAPKLLIFDTSNLYSGVPSRFSGSGSGTDFTLTISSLQPEDFATYYCYQTSRLPGFGQGTKVEIKR

SEQ ID NO: 48 - protein, 125 aa, Artificial Sequence
  synthesized_p30-097_VH
EVQLVESGGGLVQPGGSLRLSCKASGYTFTTFWMYWVWVRQAPGKGLEWVAGIDPHHGLTYYADSVKGRFTISADTSKNTAYLQMNSLRAEDTAVYYCARGDWDGLMDYWGQGTLVTVSSASAAA

SEQ ID NO: 49 - protein, 181 aa, Artificial Sequence
  synthesized_p30 protein
MKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDNETNECTSSFETLFEQEPSSEVPKDSKLYMLAQKTVQHIEQYGKAPDFNKVIRAHNFIQTIYGTPLKEEEKEVVRLMVIK

SEQ ID NO: 50 - DNA, 36 bp, Artificial Sequence
  synthesized_F primer
cgtgtcgcatctgaagtgcagctggtggaatcggga

SEQ ID NO: 51 - DNA, 48 bp, Artificial Sequence
  synthesized_R primer
gaccgatgggcccttggtgctagccgagctcacggtaacaagggtgcc

SEQ ID NO: 52 - DNA, 59 bp, Artificial Sequence
  synthesized_F primer
caggtgcacgatgtgatggtaccgatattcaaatgacccagagcccgagcagcctgagc

SEQ ID NO: 53 - DNA, 36 bp, Artificial Sequence
  synthesized_R primer
tgcagccaccgtacgtttgatttccaccttggtgcc